(19) United States
(12) Patent Application Publication
(10) Pub. No.: US 2006/0270871 A1
(43) Pub. Date: Nov. 30, 2006

(54) POLYMORPHIC FORM I OF PREGABALIN AND PROCESSES FOR ITS PREPARATION

(76) Inventors: Chandra Has Khanduri, Gurgaon (IN); Suresh Babu Jayachandra, Gurgaon (IN); Jigar Bhaskarbhai Shah, Gujarat (IN)

Correspondence Address: Jayadeep R. Deshmukh;Ranbaxy Inc., Suite 2100, 600 College Road East, Princeton, NJ, 08540, US

(21) Appl. No.: 11/420,122

(22) Filed: May 24, 2006

(30) Foreign Application Priority Data
May 30, 2005 (IN) 1386/DEL/2005

(51) Int. Cl. C07C 229/02

(52) U.S. Cl. 562/553

(57) ABSTRACT

The present invention relates to novel polymorphic Form I of pregabalin and processes for its preparation. Further provided is an enantiomerically pure pregabalin or salt thereof having less than about 0.03% w/w of the (R)-(−)-enantiomer.

### FIELD OF THE INVENTION

The present invention relates to polymorphic Form I of pregabalin and processes for its preparation. Further provided is an enantiomerically pure pregabalin or salt thereof having less than about 0.03% w/w of the (R)-(−)-enantiomer.

### CROSS-REFERENCE TO RELATED APPLICATIONS

This application claims the benefit of priority under 35 USC 119(a) of Indian application having application number 1386/DEL/2005 filed in India on May 30, 2005.

### BACKGROUND OF THE INVENTION

γ-aminobutyric acid (“GABA”) is one of the most widely distributed inhibitory neurotransmitters involved in the regulation of brain neuronal activity. The concentration of GABA is regulated by two pyridoxal 5′-phosphate dependent enzymes; L-glutamic acid decarboxylase (“GAD”), which catalyzes conversion of 1-glutamic acid to GABA and GABA aminotransferase, which degrades GABA to succinic semialdehyde. When the concentration of GABA diminishes below a threshold level in the brain, convulsions may result and, conversely, raising the GABA level appears to terminate seizures.

Pregabalin is chemically described as (S)-(+)-3-aminomethyl-5-methyl-1-hexanoic acid. It is also known as (S)-(+)-3-(2-methylpropyl)-4-aminobutanoic acid, (S)-(+)-3-isobutyl-γ-aminobutanoic acid or (S)-(+)-3-isobutyl-GABA. Pregabalin has the structure exemplified in Formula I.

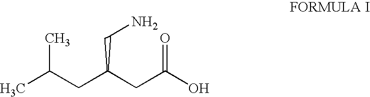

Pregabalin increases the concentration of GABA by activating GAD and therefore is useful as a therapeutic agent for the treatment of pain, convulsions, general anxiety related disorders and epileptic seizures. The (R)-(−)-enantiomer of pregabalin has been found to be about 40 times less active when compared with the (S)-(+)-enantiomer.

WO 93/23383; WO 03/093220; WO 01/55090; WO 96/38405 and WO 96/40617 provide several processes for the preparation of pregabalin. The melting point of the (S)-(+) enantiomer of pregabalin is reported to be around 177° C. to 179° C. and racemic pregabalin is reported to have a melting point around 166° C. to 167.5° C. It has also been reported that the (S)-(+)-enantiomer has a melting point between 184° C. to 186° C. None of the above-mentioned applications provide characterization data for pregabalin that include X-Ray Diffraction patterns. The moisture content of the obtained pregabalin is also not reported. Although it was reported that the maximum chiral purity of pregabalin is 100% w/w, the limit for detection of the unwanted (R)-(−)-enantiomer by the methods applied in the above-mentioned applications is 0.05% w/w. Therefore, the assertion that the obtained pregabalin obtained by the processes disclosed is 100% pure of the (R)-(−)-enantiomer cannot be verified.

### SUMMARY OF THE INVENTION

In one general aspect there is provided polymorphic crystalline Form I of pregabalin.

Embodiments of polymorphic Form I of pregabalin may include one or more of the following features. For example, the pregabalin may have a melting point of about 194° C. to about 197° C. The Form 1 of pregabalin may have an X-Ray Powder Diffraction (XRPD) pattern which includes characteristic 2θ values at 9.5, 16.62, 18.18, 18.32, 19.06, 19.74, 22.14 and 35.62. The Form I of pregabalin may further include characteristic 2θ values at 8.58, 10.02, 10.62, 10.96, 11.52, 12.26, 14.44, 17.20, 17.74, 20.14, 22.68, 23.18, 23.46, 23.88, 24.64, 26.16, 26.46, 26.90, 27.60, 27.92, 28.22, 28.58, 28.76, 29.18, 29.86, 30.24, 30.96, 31.38, 31.90, 32.88, 33.14, 33.94, 34.64, 36.42, 36.84, 36.94, 37.12, 37.40, 38.48, 38.66 and 39.54.

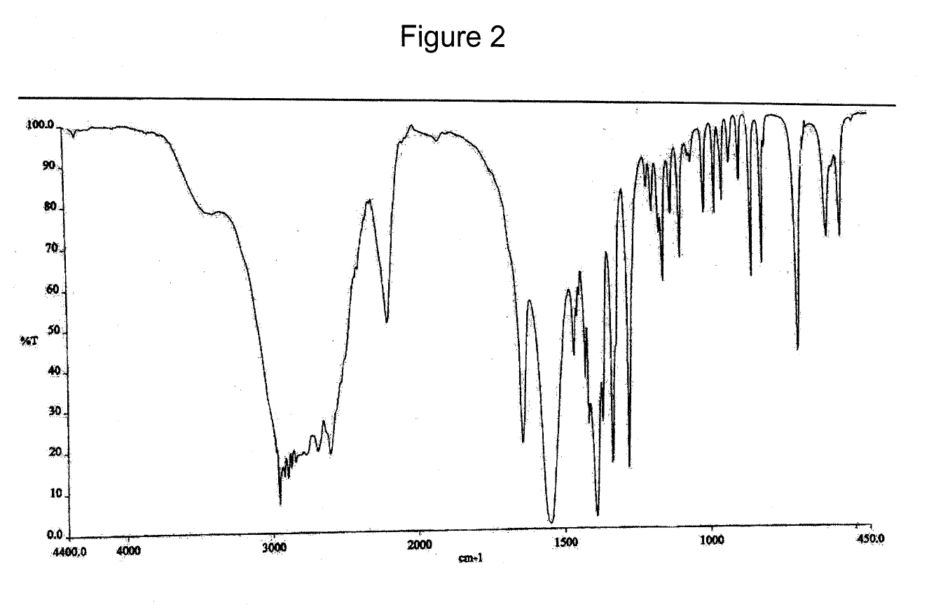

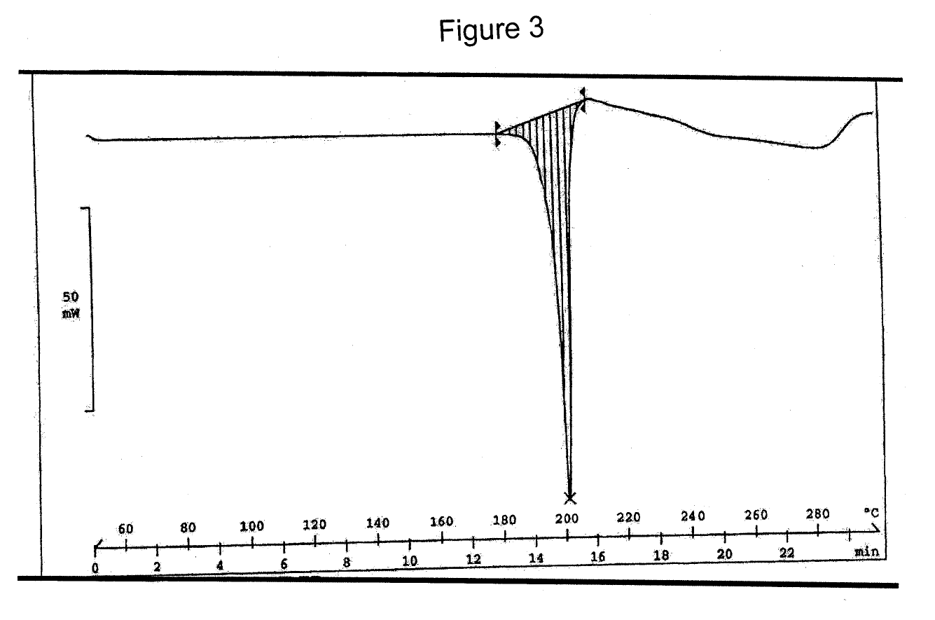

The Form I of pregabalin may have a Fourier Transform Infrared (FTIR) spectrum of Form I of pregabalin in potassium bromide as depicted in FIG. 2. The Form I of pregabalin may have a characteristic DSC thermogram as depicted in FIG. 3. The Form I of pregabalin may have a DSC thermogram showing a characteristic endothermic peak at 194° C. to 205° C. The Form I of pregabalin may be anhydrous pregabalin having a moisture content that is less than about 0.3% w/w.

In another general aspect there is provided a process for preparation of Form I of pregabalin. The process includes recrystallizing pregabalin from an aqueous alkanol solvent system wherein the water content of the reaction mass is above about 25%.

In another general aspect there is provided a pharmaceutical composition. The pharmaceutical composition includes Form I of pregabalin having an XRPD pattern comprising characteristic 2θ values at 9.5, 16.62, 18.18, 18.32, 19.06, 19.74, 22.14 and 35.62 and one or more pharmaceutically acceptable excipients.

Embodiments of the pharmaceutical composition may include one or more of the following features. For example, the pharmaceutical composition may include Form I of pregabalin which exhibits characteristic 2θ values at 8.58, 10.02, 10.62, 10.96, 11.52, 12.26, 14.44, 17.20, 17.74, 20.14, 22.68, 23.18, 23.46, 23.88, 24.64, 26.16, 26.46, 26.90, 27.60, 27.92, 28.22, 28.58, 28.76, 29.18, 29.86, 30.24, 30.96, 31.38, 31.90, 32.88, 33.14, 33.94, 34.64, 36.42, 36.84, 36.94, 37.12, 37.40, 38.48, 38.66 and 39.54. The one or more pharmaceutically acceptable excipients may be one or more of diluents, carriers, lubricants, binders, colorants, and disintegrants.

In another general aspect there is provided a method of treating pain, epilepsy, convulsions, attention deficit hypersensitivity disorder (ADHD) and general anxiety related disorders in a mammal in need thereof. The method includes administering to a mammal in need thereof a therapeutically effective quantity of Form I of pregabalin having an XRPD pattern which includes characteristic 2θ values at 9.5, 16.62, 18.18, 18.32, 19.06, 19.74, 22.14 and 35.62.

Embodiments of the method may include one or more of the following features. For example, the Form I of pregabalin may further exhibit characteristic 2θ values at 8.58, 10.02, 10.62, 10.96, 11.52, 12.26, 14.44, 17.20, 17.74, 20.14, 22.68, 23.18, 23.46, 23.88, 24.64, 26.16, 26.46, 26.90, 27.60, 27.92, 28.22, 28.58, 28.76, 29.18, 29.86, 30.24, 30.96, 31.38, 31.90, 32.88, 33.14, 33.94, 34.64, 36.42, 36.84, 36.94, 37.12, 37.40, 38.48, 38.66 and 39.54.

In another general aspect there is provided anhydrous pregabalin having a moisture content less than 0.3% w/w. Embodiments of the anhydrous pregabalin may have one or more of the features described above.

In another general aspect there is provided (S)-(+)-pregabalin or salt thereof having 0.03% or less of (R)-(−)-pregabalin or salt thereof. Embodiments of the (S)-(+)-pregabalin may have one or more of the features described above.

In another general aspect there is provided a pharmaceutical composition comprising (S)-(+)-pregabalin or salt thereof having 0.03% or less of unwanted (R)-(−)-pregabalin and one or more pharmaceutically acceptable excipients. Embodiments of the pharmaceutical compositions may have one or more of the features described above.

In another general aspect there is provided a method of treating one or more of pain, epilepsy, convulsions, attention deficit hypersensitivity disorder (ADHD) and general anxiety related disorders. The method includes administering to a mammal in need thereof a therapeutically effective quantity of (S)-(+)-pregabalin or salt thereof having 0.03% or less of unwanted (R)-(−)-pregabalin. Embodiments of the method may have one or more of the features described above.

The details of one or more embodiments of the inventions are set forth in the description below. Other features, objects and advantages of the inventions will be apparent from the description and claims.

### DETAILED DESCRIPTION OF THE DRAWINGS

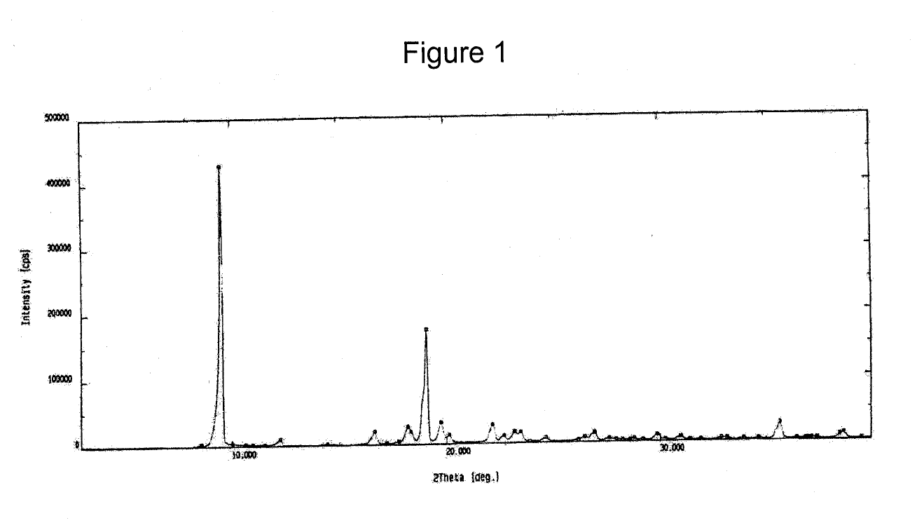

FIG. 1 provides an XRPD pattern of Form I of pregabalin.

FIG. 2 provides a FTIR spectrum of Form I of pregabalin.

FIG. 3 provides a DSC thermogram of Form I of pregabalin.

### DETAILED DESCRIPTION OF THE INVENTION

The present inventors have now surprisingly found that (S)-(+)-pregabalin may be prepared in a manner that results in a novel polymorphic form that is believed to be until now unknown. The novel polymorphic form has been designated as Form I of pregabalin. This new Form I pregabalin is characteristically different from the pregabalin known in the art as it has a melting point of about 194° C.-197° C.

The present inventors have also now prepared enantiomerically pure pregabalin or a salt thereof having less than about 0.03% w/w of the (R)-(−)-enantiomer. The limit of detection of unwanted (R)-(−)-enantiomer as per the method used by the present invention is about 0.01% w/w.

A first aspect of the invention provides for Form I of pregabalin having an XRPD pattern as depicted in FIG. 1 of the accompanying drawings. The novel polymorphic Form I pregabalin exhibits characteristic 2θ values at 9.5, 16.62, 18.18, 18.32, 19.06, 19.74, 22.14 and 35.62. The XRPD pattern of Form I of pregabalin further exhibits characteristic 2θ values at 8.58, 9.5, 10.02, 10.62, 10.96, 11.52, 12.26, 14.44, 16.62, 17.20, 17.74, 18.18, 18.32, 19.06, 19.74, 20.14, 22.14, 22.68, 23.18, 23.46, 23.88, 24.64, 26.16, 26.46, 26.90, 27.60, 27.92, 28.22, 28.58, 28.76, 29.18, 29.86, 30.24, 30.96, 31.38, 31.90, 32.88, 33.14, 33.94, 34.64, 35.62, 36.42, 36.84, 36.94, 37.12, 37.40, 38.48, 38.66 and 39.54.

The Fourier Transform Infrared (FTIR) spectrum of Form I of pregabalin in potassium bromide is depicted in FIG. 2 of the accompanying drawings. Form I of pregabalin has the characteristic DSC thermogram as depicted in FIG. 3 of the accompanying drawings. The DSC thermogram shows a characteristic endothermic peak at 194° C.-205° C.

A second aspect of the present invention provides anhydrous pregabalin having a moisture content that is less than about 0.3% w/w when determined by known techniques. The anhydrous pregabalin of the present invention is non-hygroscopic, colorless and stable during the accelerated stability testing.

A third aspect of the present invention provides (S)-(+)-pregabalin or a salt thereof having about 0.03% or less of the (R)-(−)-pregabalin or salts thereof when determined by the high pressure liquid chromatography (“HPLC”) method. Since the (R)-(−)-enantiomer is 40 times less active when compared with the (S)-(+)-pregabalin, it is desirable to have the highest possible chiral purity in the final product.

Also provided in the present invention is a process for preparation of Form I of pregabalin. The process includes the recrystallization of pregabalin from an aqueous alkanol solvent system wherein the water content of the reaction mass is above about 25% w/w.

The pregabalin is dissolved in an aqueous alkanol solvent system that includes more than about 25% w/w of water. The resultant mass is heated to reflux and cooled, which results in Form I of pregabalin precipitating out. This product may then be suitably isolated and dried.

A fifth aspect of the present invention provides a pharmaceutical composition comprising Form I of pregabalin and optionally one or more pharmaceutically acceptable excipients. Suitable pharmaceutically acceptable excipients may include one or more of diluents, carriers, lubricants, binders, colorants, and disintegrants.

A sixth aspect of the present invention provides for pharmaceutical compositions that include (S)-(+)-pregabalin or a salt thereof having about 0.03% or less of the unwanted (R)-(−)-pregabalin and, optionally, one or more pharmaceutically acceptable excipients. Suitable pharmaceutically acceptable excipients may include one or more of diluents, carriers, lubricants, binders, colorants, and disintegrants

A seventh aspect of the present invention provides a method of treating pain, epilepsy, convulsions, attention deficit hypersensitivity disorder (ADHD) and general anxiety related disorders. The method includes administering to a mammal in need thereof a therapeutically effective quantity of Form I of pregabalin.

An eighth aspect of the present invention provides a method of treating pain, epilepsy, convulsions, attention deficit hypersensitivity disorder (ADHD) and general anxiety related disorders. The method includes administering to a mammal in need thereof a therapeutically effective quantity of (S)-(+)-pregabalin or salt thereof having about 0.03% or less of the (R)-(−)-pregabalin.

Powder XRD of the samples were determined by using an X-Ray Diffractometer, Rigaku Corporation, RU-H3R, Goniometer CN2155A3, X-Ray tube with Cu target anode, Divergence slits 1 0, Receiving slit 0.15 mm, Scatter slit 1°, Power: 40 KV, 100 mA, Scanning speed: 2 deg/min step: 0.02 deg, Wave length: 1.5406 A.

FTIR of the samples were determined by using as the instrument a Perkin Elmer, 16 PC, SCAN: 16scans, 4.0 cm⁻¹, according to the USP 25, general test methods page 1920, infrared absorption spectrum by potassium bromide pellet method.

DSC thermograms were recorded using a DSC821 e, Mettler Toledo, Sample weight: 3-5 mg, Temperature range: 50° C.-350° C., Heating rate: 20° C./min, Nitrogen 80.0 mL/min, Number of holes in the crucible: 1.

The following non-limiting examples further illustrate the novel polymorphic Form I of pregabalin or a salt thereof and a process of making thereof. As such, the examples are provided merely to illustrate particular aspects of the disclosure and are not intended to limit the scope of the present invention.

### EXAMPLE 1

### 3-(AMINOMETHYL)-5-METHYLHEXANOIC ACID (RACEMIC)

A solution of potassium hydroxide (19 g) in methanol (80 ml) was added to a solution of ethyl 2-carboxyethyl-3-cyano-5-methyl hexanoate (90 g) in methanol (31 ml) at a temperature of between about 25° C. to 35° C. The mixture was heated to reflux for 4 to 5 hours, cooled to a temperature of between about 25° C. to 30° C. and a solution of potassium hydroxide (37 g) in water (62 ml) was added at a temperature of between about 30° C. to 50° C. The mass was stirred for 15 minutes and concentrated under a vacuum to about 180 ml volume. Water (30 ml) was added into the reaction mixture and further concentrated to about 160 ml. To this was added Raney Nickel (15 g wet), water (8.2 ml) and ethyl alcohol (9.2 ml) and the reaction mixture was stirred under hydrogen pressure of 3.5-4 Kg/cm² at a temperature between 35° C. and 40° C. for 24 hours.

After completion of hydrogenation, ethyl alcohol (18 ml) was added into the reaction mixture. The reaction mixture was filtered through a celite bed and the cake was washed with aqueous ethyl alcohol (110 ml). Next, glacial acetic acid (70 ml) was charged slowly at a temperature of between 40° C. and 50° C. and heated to a temperature of between 70° C. and 75° C. to dissolve the solids. The reaction mixture was slowly cooled to a temperature of between 0° C. and 5° C. and stirred at a temperature of between 0° C. and 5° C. for 4 hours. The separated product was filtered and washed with isopropanol (175 ml), then dried under a vacuum at a temperature of between 35° C. and 45° C. for 12 hours resulting in a white crystalline powder

Yield: 40 g.

HPLC Purity: 99.6%

¹HNMR (D₂O, 300 MHz): δ 0.93-0.96 (m, 6H), 1.26-1.30 (t, 2H, J=6.9 Hz), 1.7-1.74 (m, 1H), 2.2-2.4 (m, 3H), 3.0-3.07 (m, 2H).

### EXAMPLE 2

### S(+)-3-(AMINOETHYL)-5-METHYLHEXANOIC ACID (CRUDE)

A mixture of racemic 3-aminoethyl-5-methylhexanoic acid (20 g), S-(+) mandelic acid (26.6 g) and 3% water/isopropanol mixture (340 ml) was heated at a temperature between 65° C. and 70° C. to get a clear solution. The mixture was cooled to a temperature of between 40° C. and 45° C., seeded with crystals of pregabalin mandelate and then further cooled to a temperature of between 20° C. and 25° C. The mixture was stirred at a temperature of between 20° C. and 25° C. for 4 hours, the solid was filtered and washed with a 3% water/isopropanol mixture (15 ml). The wet solid was recharged into a mixture of 3% water/isopropanol mixture (70 ml) and S-(+)-mandelic acid (3.9 g). The mixture was heated to a temperature of about 65° C. and stirred for 5 minutes at a temperature of between 65° C. and 70° C. The solution was then cooled to a temperature of between 0° C. and 5° C. and stirred for 4 hours.

The separated solids were filtered and washed with a 3% water/isopropanol mixture (10 ml). The wet material was charged in a 5% water/tetrahydrofuran mixture (130 ml) and heated to a temperature of between 50° C. and 55° C. The solution was cooled to a temperature of between 0° C. and 5° C. and stirred for 4 hours. The product was filtered and washed with tetrahydrofuran (7 ml), followed by isopropanol (2×10 ml). The solid was dried under a vacuum at a temperature of between 40° C. to 45° C. for 8 hours resulting in a white solid of the title product. The yield was measured to be 5 grams.

### EXAMPLE 3

### S(+)-3-(AMINOETHYL)-5-METHYLHEXANOIC ACID (PREGABALIN)

Crude S-(+)-3-(aminoethyl)-5-methylhexanoic acid (5 g) was charged in a 30% water/isopropanol mixture (80 ml). The mixture was heated to dissolve the product at a temperature of between 75° C. and 80° C. The reaction mixture was filtered while hot, and then cooled to a temperature of between 0° C. and 5° C. The mass was further stirred for 4 hours at 0° C. to 5° C., filtered and the cake washed with chilled isopropanol (20 ml). The wet material was dried under a vacuum at a temperature of between 35° C. to 45° C. for 12 hours resulting in a white crystalline solid.

Yield: 4.8 g

HPLC Purity: 99.7% w/w

Chiral Purity: 99.5% (S-isomer), 0.024% (R-isomer)

Water content: 0.03% w/w (Karl Fischer analysis)

¹H-NMR (D₂O, 300 MHz): δ 0.89-0.93 (m, 6H), 1.21-1.26 (t, 2H, J=7.1 Hz), 1.65-1.7 (m, 1H), 2.15-2.36 (m, 3H), 2.97-3.02 (m, 2H)

While the present invention has been described in terms of its specific embodiments, certain modifications and equivalents will be apparent to those skilled in the art and are included within the scope of the present invention.

We claim:

1. Polymorphic crystalline Form 1 of pregabalin.

2. The Form 1 of pregabalin of claim 1, wherein the pregabalin has a melting point of about 194° C.-197° C.

3. The Form I of pregabalin of claim 1 having an X-Ray Powder Diffraction (XRPD) pattern comprising characteristic 2θ values at 9.5, 16.62, 18.18, 18.32, 19.06, 19.74, 22.14 and 35.62.

4. The Form I of pregabalin of claim 3, further comprising characteristic 2θ values at 8.58, 10.02, 10.62, 10.96, 11.52, 12.26, 14.44, 17.20, 17.74, 20.14, 22.68, 23.18, 23.46, 23.88, 24.64, 26.16, 26.46, 26.90, 27.60, 27.92, 28.22, 28.58, 28.76, 29.18, 29.86, 30.24, 30.96, 31.38, 31.90, 32.88, 33.14, 33.94, 34.64, 36.42, 36.84, 36.94, 37.12, 37.40, 38.48, 38.66 and 39.54.

5. The Form I of pregabalin of claim 1 having a Fourier Transform Infrared (FTIR) spectrum of Form I of pregabalin in potassium bromide as depicted in FIG. 2.

6. The Form I of pregabalin of claim 5 having the characteristic DSC thermogram as depicted in FIG. 3.

7. The Form I of pregabalin of claim 1 having a DSC thermogram showing a characteristic endothermic peak at 194° C.-205° C.

8. The Form I of pregabalin of claim 1, wherein the pregabalin is anhydrous pregabalin having a moisture content that is less than about 0.3% w/w.

9. A process for the preparation of Form I of pregabalin, the process comprising recrystallizing pregabalin from an aqueous alkanol solvent system wherein the water content of the reaction mass is above about 25%.

10. A pharmaceutical composition comprising Form I of pregabalin having an X-Ray Powder Diffraction (XRPD) pattern comprising characteristic 2θ values at 9.5, 16.62, 18.18, 18.32, 19.06, 19.74, 22.14 and 35.62 and one or more pharmaceutically acceptable excipients.

11. The pharmaceutical composition of claim 10, wherein the Form I of pregabalin further comprises characteristic 2θ values at 8.58, 10.02, 10.62, 10.96, 11.52, 12.26, 14.44, 17.20, 17.74, 20.14, 22.68, 23.18, 23.46, 23.88, 24.64, 26.16, 26.46, 26.90, 27.60, 27.92, 28.22, 28.58, 28.76, 29.18, 29.86, 30.24, 30.96, 31.38, 31.90, 32.88, 33.14, 33.94, 34.64, 36.42, 36.84, 36.94, 37.12, 37.40, 38.48, 38.66 and 39.54.

12. The pharmaceutical composition of claim 10, wherein the one or more pharmaceutically acceptable excipients comprise one or more of diluents, carriers, lubricants, binders, colorants, and disintegrants.

13. A method of treating one or more of pain, epilepsy, convulsions, attention deficit hypersensitivity disorder (ADHD) and general anxiety related disorders, the method comrpising administering to a mammal in need thereof a therapeutically effective quantity of Form I of pregabalin having an X-Ray Powder Diffraction (XRPD) pattern comprising characteristic 2θ values at 9.5, 16.62, 18.18, 18.32, 19.06, 19.74, 22.14 and 35.62.

14. The method of claim 13, wherein the Form I of pregabalin further comprises characteristic 2θ values at 8.58, 10.02, 10.62, 10.96, 11.52, 12.26, 14.44, 17.20, 17.74, 20.14, 22.68, 23.18, 23.46, 23.88, 24.64, 26.16, 26.46, 26.90, 27.60, 27.92, 28.22, 28.58, 28.76, 29.18, 29.86, 30.24, 30.96, 31.38, 31.90, 32.88, 33.14, 33.94, 34.64, 36.42, 36.84, 36.94, 37.12, 37.40, 38.48, 38.66 and 39.54.

15. Anhydrous pregabalin having moisture content less than 0.3% w/w.

16. (S)-(+)-pregabalin or salt thereof having 0.03% or less of (R)-(−)-pregabalin or salt thereof.

17. A pharmaceutical composition comprising (S)-(+)-pregabalin or salt thereof having 0.03% or less of unwanted (R)-(−)-pregabalin and one or more pharmaceutically acceptable excipients.

18. A method of treating one or more of pain, epilepsy, convulsions, attention deficit hypersensitivity disorder (ADHD) and general anxiety related disorders, the method comprising administering to a mammal in need thereof a therapeutically effective quantity of (S)-(+)-pregabalin or salt thereof having 0.03% or less of unwanted (R)-(−)-pregabalin.
